(19) United States
(12) Patent Application Publication
(10) Pub. No.: US 2025/0177276 A1
(43) Pub. Date: Jun. 5, 2025

(54) COMPOSITION FOR CELLULAR DNA DAMAGE REPAIR COMPRISING KAEMPFEROL SACCHARIDES COMPOUNDS

(71) Applicant: AMOREPACIFIC CORPORATION, Seoul (KR)

(72) Inventors: Nok Hyun PARK, Yongin-si (KR); Eun Soo LEE, Yongin-si (KR); Yongjoo NA, Yongin-si (KR); Jaeyoung KO, Yongin-si (KR); Saitbyul PARK, Yongin-si (KR)

(73) Assignee: AMOREPACIFIC CORPORATION, Seoul (KR)

(21) Appl. No.: 18/960,938

(22) Filed: Nov. 26, 2024

(30) Foreign Application Priority Data
Nov. 30, 2023 (KR) 10-2023-0171480

(51) Int. Cl. A61K 8/60; A61Q 17/04; A61Q 19/00

(52) U.S. Cl. CPC A61K 8/602; A61Q 17/04; A61Q 19/004; A61K 2800/92

(57) ABSTRACT

The present disclosure provides a composition for repairing cellular DNA damage which contains a kaempferol compound extracted from Camellia japonica seed cake, which is discarded after extracting oil from Camellia japonica seed, as an active ingredient, and which is nature-friendly, is safe for human body with few side effects, and can protect damaged DNA or cells from photo-stimulation.

### CROSS REFERENCE TO RELATED APPLICATION

The present application claims priority to Korean Patent Application No. 10-2023-0171480, filed on Nov. 30, 2023, the entire contents of which is incorporated herein for all purposes by this reference.

### BACKGROUND OF THE INVENTION

### Field of the Invention

The present specification discloses a composition for repairing cellular DNA damage, which comprises a kaempferol saccharide compound, is safe for the human body, and can repair DNA or skin cells damaged by light stimulation such as ultraviolet rays.

### Description of the Related Art

Aging occurs due to various causes. Among them, ultraviolet rays are mentioned as the most common cause, and accordingly, there are many reports on skin photoaging caused by ultraviolet rays. When photodamage by ultraviolet rays occurs, damage occurs to the DNA in the nucleus of cells, which causes various cell damages (cell death, cell aging, metabolic problems, etc.).

Specifically, ultraviolet rays are classified into UVA, UVB, and UVC according to wavelength, and among them, UVA has a long wavelength of 320 to 400 nm, and can reach the dermis of the skin and cause skin irritation if exposed to the skin for a long time. UVB has a medium wavelength of 290 to 320 nm, and can reach the epidermis or upper dermis layer of the skin, causing skin irritation in a short period of time.

Therefore, a substance is needed to help recover DNA, cells, and especially skin cells damaged by ultraviolet rays stimulation of UVA and UVB.

### SUMMARY OF THE INVENTION

In one aspect of the present disclosure, it is intended to provide a composition capable of repairing cellular DNA damage, which comprises a kaempferol saccharides compound as an active ingredient, so that it is safe for the human body with few side effects.

In another aspect of the present disclosure, it is intended to provide a composition for repairing cellular DNA damage utilizing waste resources discarded after extracting Camellia japonica seed oil, which includes a kaempferol saccharide compound extracted from Camellia japonica seed cake as an active ingredient.

The present specification provides a composition for repairing cellular DNA damage, comprising a kaempferol saccharides compound represented by any one of the following Chemical Formulas 1 to 8, an isomer thereof, a pharmaceutically acceptable salt thereof, a hydrate thereof, or a solvate thereof, as an active ingredient.

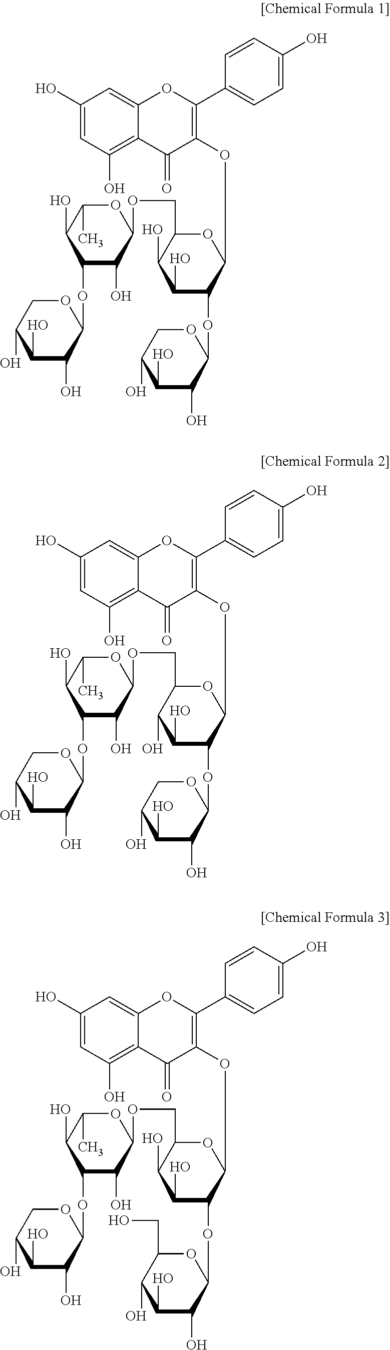

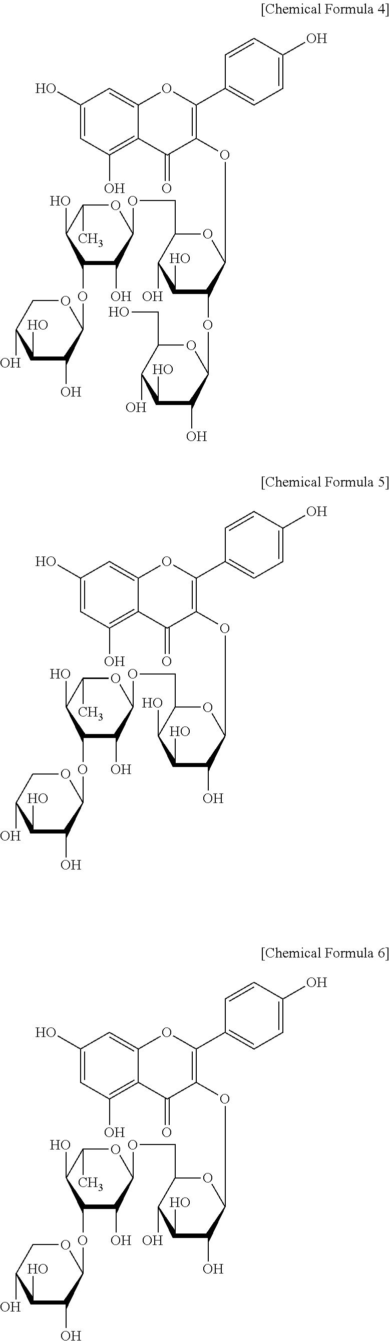

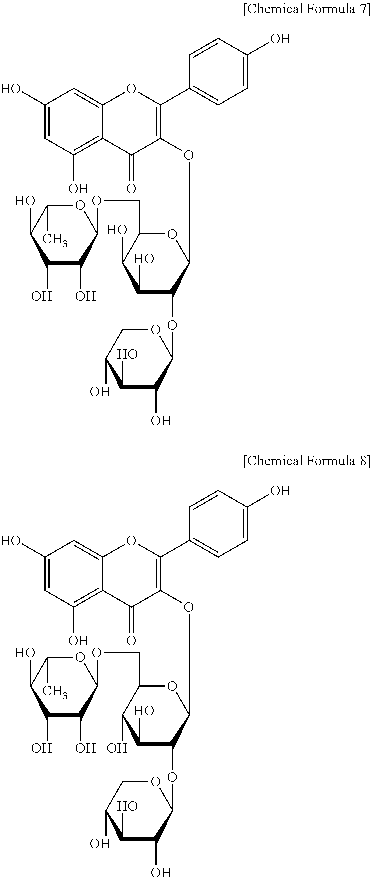

In one aspect, the composition for repairing cellular DNA damage disclosed herein comprises a kaempferol 3 or 4 saccharides compound as an active ingredient, which can repair DNA or skin cells damaged by photo-stimulation.

### BRIEF DESCRIPTION OF THE DRAWINGS

FIG. 1 illustrates a sensitive molecular marker for DNA damage and repair according to an embodiment of the present disclosure.

FIG. 2 illustrates DNA damage inhibition efficacy by γ-H2AX expression evaluation imaging according to an embodiment of the present disclosure.

FIG. 3 illustrates DNA damage inhibition efficacy by quantitative analysis of γ-H2AX expression intensity according to an embodiment of the present disclosure.

FIG. 4 illustrates cell viability imaging by ultraviolet rays according to an embodiment of the present disclosure.

FIG. 5 illustrates cell number analysis by quantitative analysis of cell viability by ultraviolet rays according to an embodiment of the present disclosure.

FIG. 6 illustrates NMR information of compounds of Chemical Formulas 1 to 4 according to an embodiment of the present disclosure.

FIG. 7 illustrates NMR information of compounds of Chemical Formulas 5 to 8 according to an embodiment of the present disclosure.

### DETAILED DESCRIPTION OF THE INVENTION

In the present specification, a description of a certain part “including” certain constituents means capable of further including other constituents, and does not exclude other constituents unless particularly stated on the contrary.

Hereinafter, the present disclosure will be described in more detail.

An exemplary embodiment of the present disclosure provides a composition for repairing cellular DNA damage, comprising a kaempferol saccharide compound represented by any one of the following Chemical Formulas 1 to 8, an isomer thereof, a pharmaceutically acceptable salt thereof, a hydrate thereof, or a solvate thereof, as an active ingredient.

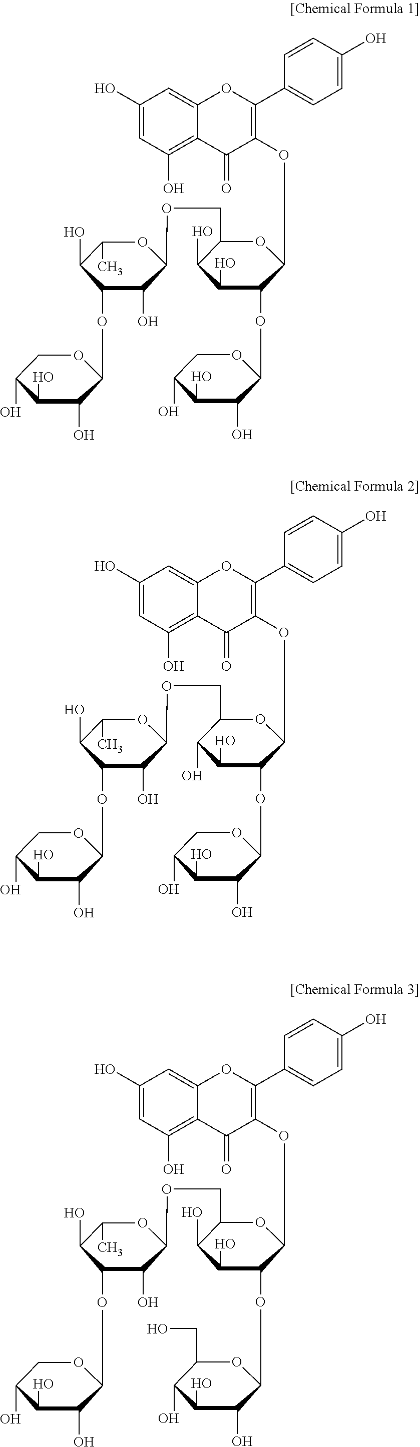

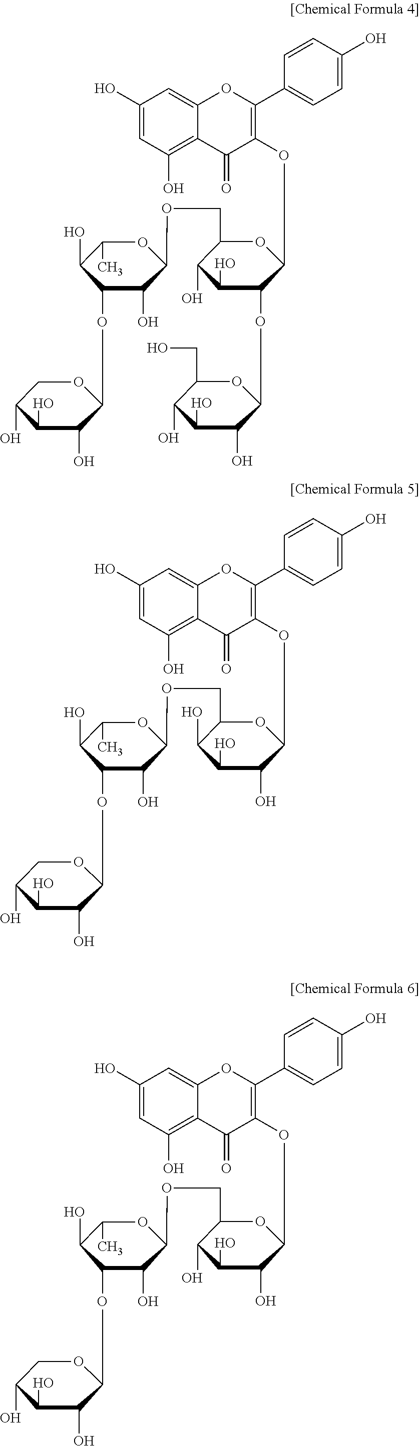

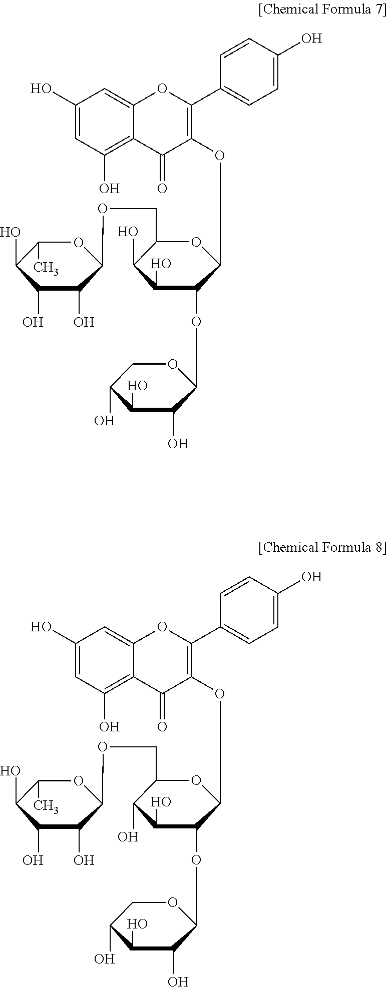

According to the present disclosure, through skin cell DNA repair, not only can already damaged skin cells be recovered, but skin that is about to be damaged can also be prevented from being damaged in advance through DNA repair.

Another exemplary embodiment of the present disclosure provides a method for repairing cellular DNA damage, comprising administering or applying the composition described above in an effective amount to a subject in need thereof.

Still another exemplary embodiment of the present disclosure provides a use of a kaempferol saccharide compound represented by any one of the following Chemical Formulae 1 to 8, an isomer thereof, a pharmaceutically acceptable salt thereof, a hydrate thereof, or a solvate thereof for preparing a composition for repairing cellular DNA damage described above.

Still another exemplary embodiment of the present disclosure provides a non-therapeutic use of a kaempferol saccharide compound represented by any one of the following Chemical Formulae 1 to 8, an isomer thereof, a pharmaceutically acceptable salt thereof, a hydrate thereof, or a solvate thereof for repairing cellular DNA damage.

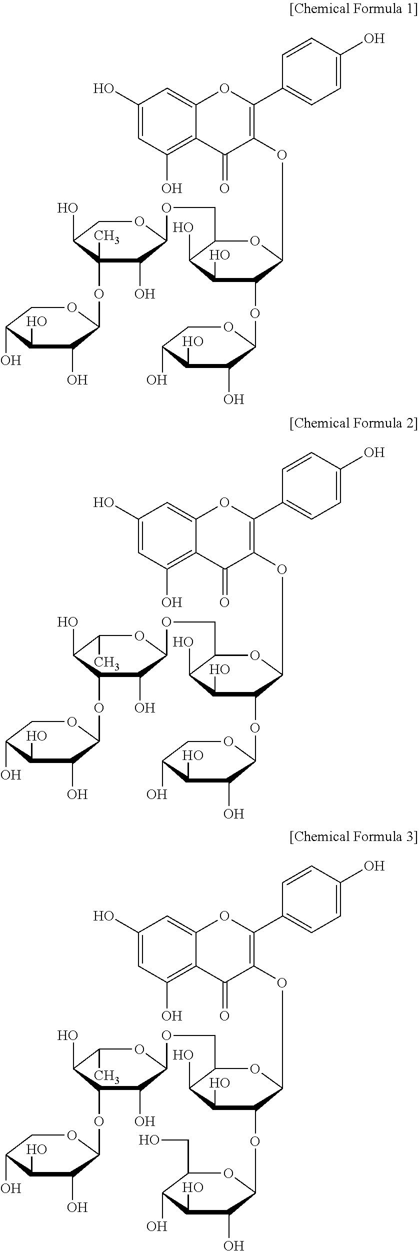

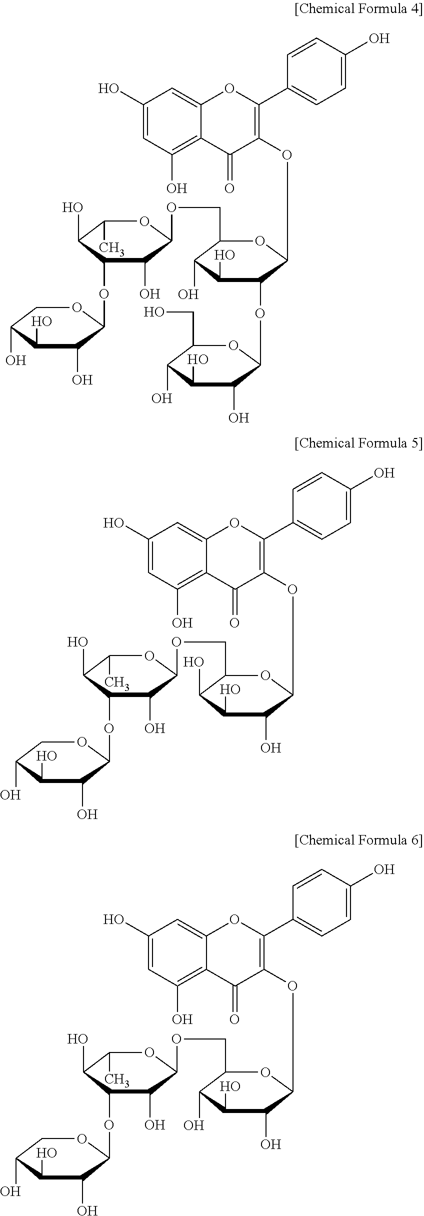

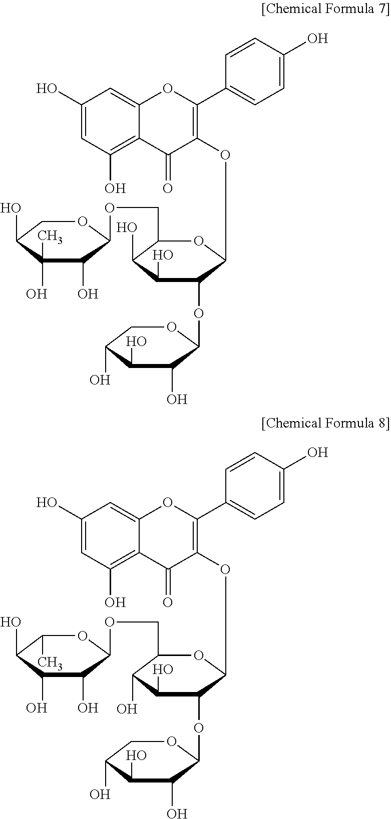

Another exemplary embodiments of the present disclosure provides a kaempferol saccharide compound represented by any one of the following Chemical Formulae 1 to 8, an isomer thereof, a pharmaceutically acceptable salt thereof, a hydrate thereof, or a solvate thereof, for use in alleviating, preventing, or treating a disease induced by photo-stimulation is provided.

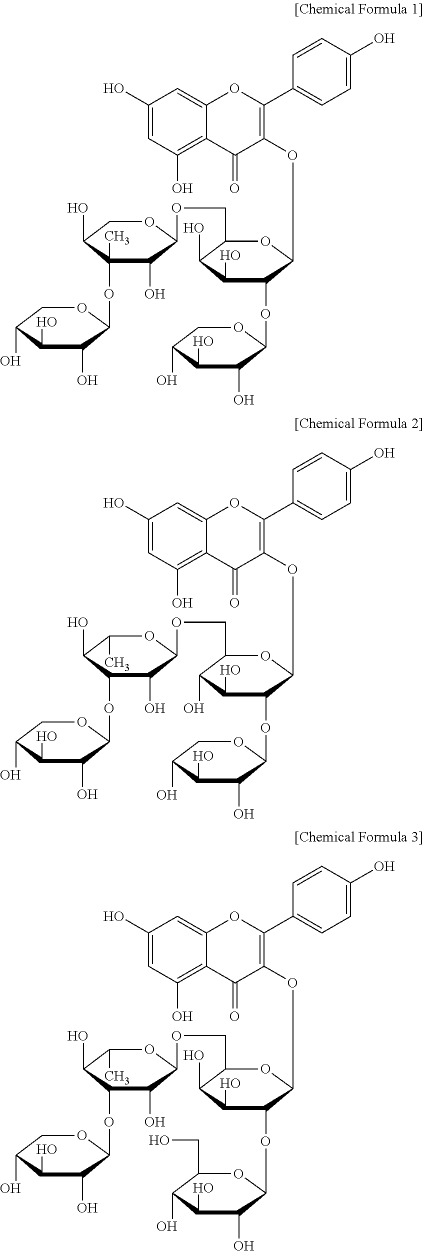

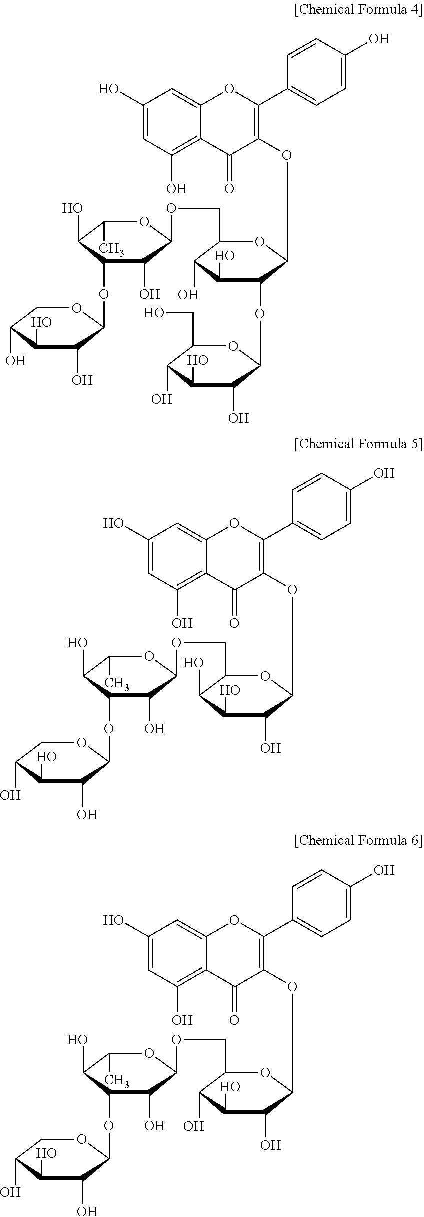

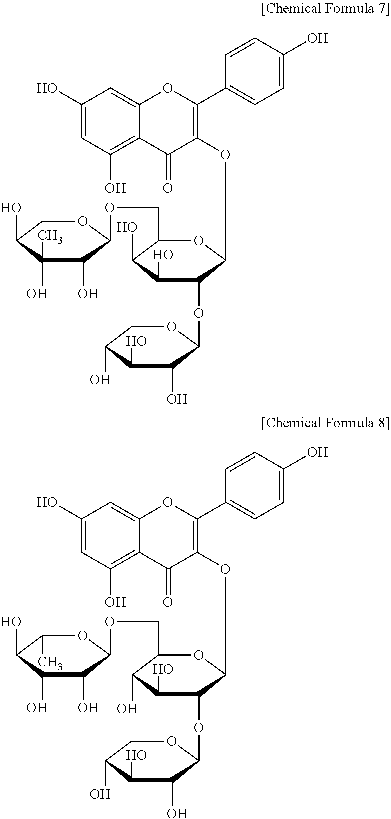

In the present disclosure, the “isomer” particularly includes not only optical isomers (e.g., essentially pure enantiomers, essentially pure diastereomers or mixtures thereof) but also conformational isomers (i.e., isomers differing only in the angle of one or more chemical bond), position isomers (particularly tautomers) or geometric isomers (e.g., cis-trans isomers).

In the present disclosure, “essentially pure” means that, when use in connection with, for example, enantiomers or diastereomers, a specific compound is present at about 90% (w/w) or more, specifically about 95% or more, more specifically about 97% or more or about 98% or more, even more specifically about 99% or more, yet more specifically about 99.5% or more.

In the present disclosure, “pharmaceutically acceptable” means being recognized as being approvable or approved by the government or a regulatory agency equivalent thereto for use in animals, more particularly in human, by avoiding significant toxic effects when used in conventional medicinal dosages or as being listed or described in the Pharmacopeia or other general pharmacopeias.

In the present disclosure, the “pharmaceutically acceptable salt” means a salt according to an aspect of the present disclosure which is pharmaceutically acceptable and has the desired pharmacological activity of the parent compound. The salt may include: (1) an acid addition salt formed from an inorganic acid such as hydrochloric acid, hydrobromic acid, sulfuric acid, nitric acid, phosphoric acid, etc.; or an organic acid such as acetic acid, priopionic acid, hexanoic acid, cyclopentylpropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, ethane-1,2-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2,2,2]-oct-2-ene-1-carboxylic acid, glucoheptonic acid, 3-phenylpropionic acid, trimethylacetic acid, tert-butylacetic acid, lauryl sulfuric acid, gluconic acid, glutamic acid, hydroxynaphthoic acid, salicylic acid, stearic acid or muconic acid; or (2) a salt formed when an acidic proton present in the parent compound is replaced

In the present disclosure, the “hydrate” means a compound to which water is bound and is used in a broad concept, including an inclusion compound lacking chemical bonding between water and the compound.

In the present disclosure, the “solvate” means a higher-order compound formed from a molecule or ion of a solute and a molecule or ion of a solvent.

Camellia japonica seed cake extract contains terpene saccharides, flavonoid saccharides, sugars and proteins. In particular, the flavonoid saccharides of the Camellia japonica seed cake include kaempferol tetrasaccharides (Chemical Formulas 1, 2, 3 and 4) and trisaccharides (Chemical Formulas 5, 6, 7 and 8).

The IUPAC name of the kaempferol saccharide compound represented by Chemical Formula 1 is Kaempferol-3-O-β-D-xylopyranosyl-(1→3)-O-α-L-rhamnopyranosyl-(1->6)-O-β-D-xylopyranosyl-(1→2)-O-β-D-galactopyranoside. It can exist as pale yellow powder at room temperature.

The information of the kaempferol saccharide compound represented by Chemical Formula 1 is as follows.

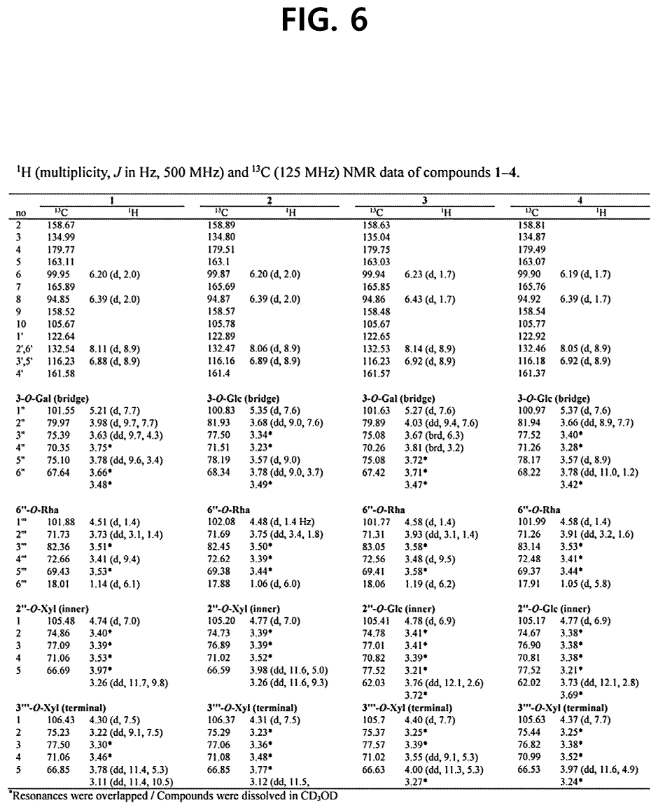

Pale yellow powder; UV (HPLC-PDA) λₘₐₓ 266, 347 nm; [α]²²_{D} −57.4 (c 0.10); ESI-Q-TOF-MS: m/z 859.2503 [M+H]⁺ (calcd. for C₃₇H₄₇O₂₃⁺, 859.2508), 881.2329 [M+Na]⁺ (calcd. for C₃₇H₄₆O₂₃Na⁺, 859.2327); ¹H- (CD₃OD, 500 MHz) and ¹³C-NMR (CD₃OD, 125 MHz) (See FIG. 6)

The IUPAC name of the kaempferol saccharide compound represented by Chemical Formula 2 is Kaempferol-3-O-β-D-xylopyranosyl-(1→3)-O-α-L-rhamnopyranosyl-(1→6)-O-β-D-xylopyranosyl-(1→2)-O-β-D-glucopyranoside. It can exist as pale yellow powder at room temperature.

The information of the kaempferol saccharide compound represented by Chemical Formula 2 is as follows.

Pale yellow powder; UV (HPLC-PDA) λₘₐₓ 266, 347 nm; [α]₂₂^{D} −63.7 (c 0.10); ESI-Q-TOF-MS: m/z 859.2505 [M+H]⁺ (calcd. for C₃₇H₄₇O₂₃⁺, 859.2508), 881.2325 [M+Na]⁺ (calcd. for C₃₇H₄₆O₂₃Na⁺, 859.2327); ¹H- (CD₃OD, 500 MHz) and ¹³C-NMR (CD₃OD, 125 MHZ) (See FIG. 6)

The IUPAC name of the kaempferol saccharide compound represented by Chemical Formula 3 is Kaempferol-3-O-β-D-xylopyranosyl-(1→3)-O-α-L-rhamnopyranosyl-(1→6)-O-β-D-glucopyranosyl-(1→2)-O-β-D-galactopyranoside. It can exist as pale yellow powder at room temperature.

The information of the kaempferol saccharide compound represented by Chemical Formula 3 is as follows.

Pale yellow powder; UV (HPLC-PDA) λₘₐₓ 266, 347 nm; [α]₂₂^{D} −72.5 (c 0.2); ESI-Q-TOF-MS: m/z 889.2616 [M+H]⁺ (calcd. for C₃₈H₄₉O₂₃⁺, 889.2613) and 911.2432 [M+Na]⁺ (calcd. for C₃₈H₄₈O₂₄Na⁺, 859.2327); ¹H- (CD₃OD, 500 MHz) and ¹³C-NMR (CD₃OD, 125 MHz) (See FIG. 6)

The IUPAC name of the kaempferol saccharide compound represented by Chemical Formula 4 is Kaempferol-3-O-β-D-xylopyranosyl-(1→3)-O-α-L-rhamnopyranosyl-(1→6)-O-β-D-glucopyranosyl-(1→2)-O-β-D-gluctopyranoside. It can exist as pale yellow powder at room temperature.

The information of the kaempferol saccharide compound represented by Chemical Formula 4 is as follows.

Pale yellow powder; UV (HPLC-PDA) λₘₐₓ 266, 347 nm; [α]₂₂^{D} −45.1 (c 0.10); ESI-Q-TOF-MS: m/z 889.2613 [M+H]⁺ (calcd. for C₃₈H₄₉O₂₃⁺, 889.2613) and 911.2423 [M+Na]⁺ (calcd. for C₃₈H₄₈O₂₄Na⁺, 859.2327); ¹H- (CD₃OD, 500 MHz) and ¹³C-NMR (CD₃OD, 125 MHz) (See FIG. 6)

A composition according to one aspect of the present disclosure comprises, as an active ingredient, Kaempferol trisaccharides or Kaempferol tetrasaccharides extracted from Camellia japonica seed cake discarded after oil extraction from Camellia japonica seeds, and thus can exhibit a significantly superior cell protection effect compared to compositions comprising conventional kaempferol saccharide compounds as active ingredients.

The IUPAC name of the kaempferol saccharide compound represented by Chemical Formula 5 is Kaempferol-3-O-β-D-xylopyranosyl-(1→3)-O-α-L-rhamnopyranosyl-(1→6)-O-β-D-galactopyranoside. It can exist as pale yellow powder at room temperature.

The information of the kaempferol saccharide compound represented by Chemical Formula 5 is as follows.

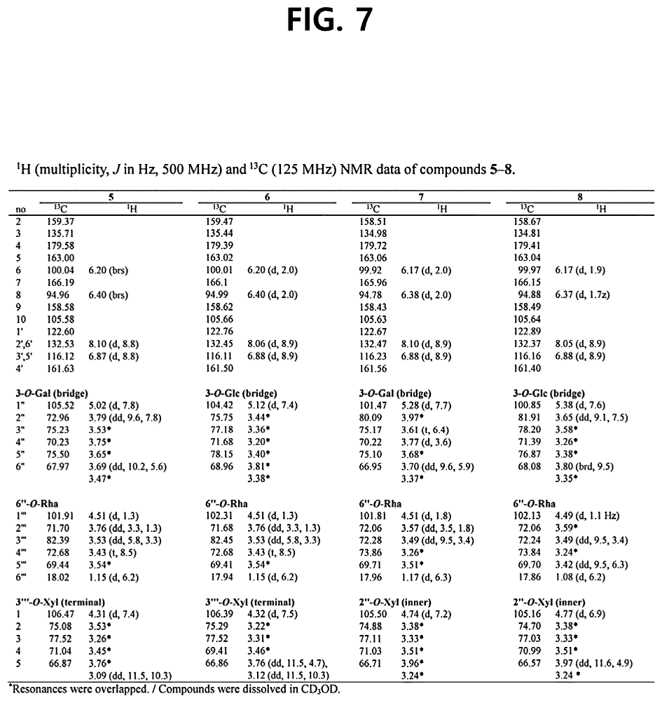

Pale yellow powder; UV (HPLC-PDA) λₘₐₓ 265, 346 nm; [α]²²_{D} −62.1 (c 0.10); ESI-Q-TOF-MS: m/z 727.2904 [M+H]⁺ (calcd. for C₃₂H₃₉O₁₉₇, 727.2086) and 749.1915 [M+Na]⁺ (calcd. for C₃₂H₃₈O₁₉Na⁺, 749.1905); ¹H- (CD₃OD, 500 MHz) and ¹³C-NMR (CD₃OD, 125 MHz) (See FIG. 7)

The IUPAC name of the kaempferol saccharide compound represented by Chemical Formula 6 is Kaempferol-3-O-β-D-xylopyranosyl-(1→3)-O-α-L-rhamnopyranosyl-(1→6)-O-β-D-glucopyranoside. It can exist as pale yellow powder at room temperature.

The information of the kaempferol saccharide compound represented by Chemical Formula 6 is as follows.

Pale yellow powder; UV (HPLC-PDA) λₘₐₓ 265, 346 nm; [α]²²_{D} 53.9 (c 0.10); ESI-Q-TOF-MS: m/z 727.2097 [M+H]⁺ (calcd. for C₃₂H₃₉O₁₉⁺, 727.2686); ¹H- (CD₃OD, 500 MHz) and ¹³C-NMR (CD₃OD, 125 MHz) (See FIG. 7)

The IUPAC name of the kaempferol saccharide compound represented by Chemical Formula 7 is Kaempferol-3-O-α-L-rhamnopyranosyl-(1→6)-O-β-D-xylopyranosyl-(1→2)-O-β-D-galactopyranoside. It can exist as pale yellow powder at room temperature.

The information of the kaempferol saccharide compound represented by Chemical Formula 7 is as follows.

Pale yellow powder; UV (HPLC-PDA) λₘₐₓ 265, 346 nm; [α]₂₂^{D} −70.8 (c 0.15); ESI-Q-TOF-MS: m/z 727.2108 [M+H]⁺ (calcd. for C₃₂H₃₉O₁₉₄, 727.2086) and 749.1916 [M+Na]⁺ (calcd. for C₃₂H₃₈O₁₉Na⁺, 749.1905); ¹H- (CD₃OD, 500 MHz) and ¹³C-NMR (CD₃OD, 125 MHz) (See FIG. 7)

The IUPAC name of the kaempferol saccharide compound represented by Chemical Formula 8 is Kaempferol-3-O-α-L-rhamnopyranosyl-(1→6)-O-β-D-xylopyranosyl-(1→2)-O-β-D-glucopyranoside. It can exist as pale yellow powder at room temperature.

The information of the kaempferol saccharide compound represented by Chemical Formula 8 is as follows.

Pale yellow powder; UV (HPLC-PDA) λₘₐₓ 265, 346 nm; [α]₂₂^{D} −39.6 (c 0.10); ESI-Q-TOF-MS: m/z 749.1902 [M+Na]⁺ (calcd. for C₃₂H₃₈O₁₉Na⁺, 749.1905); ¹H- (CD₃OD, 500 MHz) and ¹³C-NMR (CD₃OD, 125 MHz) (See FIG. 7)

In an exemplary embodiment, the kaempferol saccharide compound may be one extracted from Camellia japonica seed cake.

Specifically, the kaempferol saccharide compound may be obtained by obtaining Camellia japonica seed cake which is obtained after producing oil from Camellia japonica seed through compression and then extracting a kaempferol saccharide compound from the Camellia japonica seed cake using 70% ethanol.

In an embodiment, the concentration of the active ingredient may be 0.01 to 100 μM, for example, 0.01 μM or higher, 0.1 μM or higher, 0.5 μM or higher, 1 μM or higher, 1.5 μM or higher, 2 μM or higher or 2.5 μM or higher, and 100 μM or lower, 50 μM or lower, 10 μM or lower, 8 μM or lower, 6 μM or lower or 4 μM or lower, based on the total weight of the composition.

In an embodiment, the cell may be a skin cell.

In an embodiment, the composition may be for inhibiting or preventing skin damage.

In an embodiment, the composition may be for improving skin's natural regenerative power.

In an embodiment, the composition may inhibit the expression of a DNA damage gene.

In an embodiment, the DNA damage gene may be γH2AX.

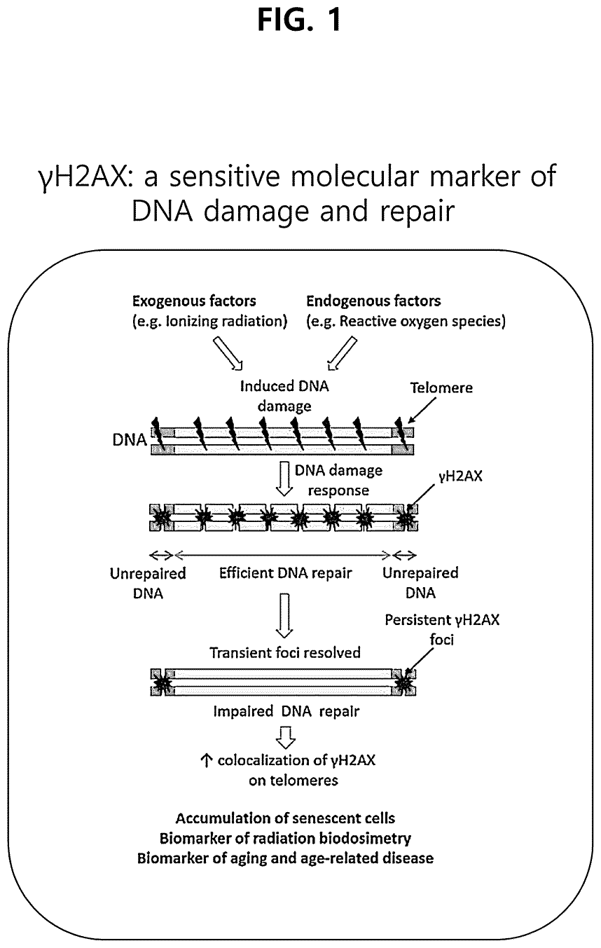

For example, when cells are exposed to ultraviolet rays, the DNA in the nucleus of the cell is damaged and the cell tries to repair it on its own. However, if the damage is severe and repair is not possible, γH2AX is created at the Ser-139 residue of histone H2AX in DNA. Therefore, γH2AX is used as a very well-known DNA damage marker. (See FIG. 1)

In an embodiment, the damage may be damage caused by ultraviolet rays.

In an embodiment, the ultraviolet rays may be at least one of UVA and UVB.

In an embodiment, the composition may be a cosmetic composition.

The cosmetic composition according to an embodiment of the present disclosure may contain a cosmetologically or dermatologically acceptable medium or base. It may be prepared into any topically applicable formulation, for example, in the form of a solution, a gel, a solid, an anhydrous paste, an oil-in-water emulsion, a suspension, a microemulsion, a microcapsule, a microgranule, an ionic (liposomal) or nonionic vesicular dispersion, a cream, a lotion, a powder, an ointment, a spray or a concealer stick. These compositions may be prepared according to common methods in the art. The composition according to the present disclosure may also be used in the form of a foam or an aerosol composition further containing a compressed propellant.

The cosmetic composition according to an embodiment of the present disclosure is not particularly limited in its formulation. For example, it may be formulated into cosmetics such as a softening lotion, an astringent lotion, a nourishing lotion, a nourishing cream, a massage cream, an essence, an eye cream, an eye essence, a cleansing cream, a cleansing foam, a cleansing water, a pack, a powder, a body lotion, a body cream, a body oil, a body essence, etc.

When the formulation according to an embodiment of the present disclosure is a paste, a cream or a gel, animal fiber, plant fiber, wax, paraffin, starch, tragacanth, a cellulose derivative, polyethylene glycol, silicon, bentonite, silica, talc, zinc oxide, etc. may be used as a carrier ingredient.

When the formulation according to an embodiment of the present disclosure is a powder or a spray, lactose, talc, silica, aluminum hydroxide, calcium silicate or polyamide powder may be used as a carrier ingredient. Particularly, a spray may additionally contain a propellant such as chlorofluorohydrocarbon, propane/butane or dimethyl ether.

When the formulation according to an embodiment of the present disclosure is a solution or an emulsion, a solvent, solubilizer or an emulsifier may be used as a carrier ingredient. Examples include water, ethanol, isopropanol, ethyl carbonate, ethyl acetate, benzyl alcohol, benzyl benzoate, propylene glycol, 1,3-butylene glycol, aliphatic glycerol ester, polyethylene glycol or a fatty acid ester of sorbitan.

When the formulation according to an embodiment of the present disclosure is a suspension, a liquid diluent such as water, ethanol or propylene glycol, a suspending agent such as ethoxylated isostearyl alcohol, polyoxyethylene sorbitol ester and polyoxyethylene sorbitan ester, microcrystalline cellulose, aluminum metahydroxide, bentonite, agar, tragacanth, etc. may be used as a carrier ingredient.

When the formulation according to an embodiment of the present disclosure is a surfactant-containing cleanser, aliphatic alcohol sulfate, aliphatic alcohol ether sulfate, sulfosuccinic monoester, isethionate, an imidazolinium derivative, methyl taurate, sarcosinate, a fatty acid amide ether sulfate, an amidoalkyl betaine, an aliphatic alcohol, a fatty acid glyceride, a fatty acid diethanolamide, vegetable oil, a lanolin derivative, an ethoxylated glycerol fatty acid ester, etc. may be used as a carrier ingredient.

The cosmetic composition according to an embodiment of the present disclosure may further contain, in addition to the active ingredient, a functional additive and ingredients contained in general cosmetic compositions. The functional additive may be an ingredient selected from a group consisting of a water-soluble vitamin, an oil-soluble vitamin, a polypeptide, a polysaccharide, a sphingolipid and a seaweed extract.

In addition, the cosmetic composition according to an embodiment of the present disclosure may further contain ingredients contained in general cosmetic compositions together with the functional additive, if necessary. As the further contained ingredient, there may be an oil or fat, a humectant, an emollient, a surfactant, an organic and inorganic pigments, an organic powder, a UV absorbent, an antiseptic, a sterilizer, an antioxidant, a plant extract, a pH control agent, an alcohol, a colorant, a flavorant, a blood circulation stimulant, a cooling agent, an antiperspirant, purified water, etc.

In addition, the composition according to an embodiment of the present disclosure may be a composition for external application to skin. The composition for external application to skin collectively refers to any form that can be applied externally to skin, and various formulations of cosmetics and pharmaceuticals may be included therein.

In an embodiment, the composition may be a pharmaceutical composition.

In an embodiment, the composition may be a pharmaceutical composition for alleviating, preventing or treating a disease caused by photo-stimulation.

The pharmaceutical composition according to an embodiment of the present disclosure may further contain a pharmaceutical adjuvant such as an antiseptic, a stabilizer, a wetting agent, an emulsification promoter, a salt and/or a buffer for controlling osmotic pressure, etc. and other therapeutically useful substances, and may be formulated into various forms for oral administration or parenteral administration.

The formulation for oral administration may be, for example, a tablet, a pill, a hard or soft capsule, a liquid, a suspension, an emulsion, a syrup, a powder, a dust, a fine granule, a granule, a pellet, etc., and these formulations may contain, in addition to the active ingredient, a surfactant, a diluent (e.g., lactose, dextrose, sucrose, mannitol, sorbitol, cellulose or glycine) or a lubricant (e.g., silica, talc, stearic acid and its magnesium or calcium salt or polyethylene glycol). The tablet may further contain a binder such as magnesium aluminum silicate, starch paste, gelatin, tragacanth, methylcellulose, sodium carboxymethylcellulose and polyvinylpyrrolidone and may contain, in some cases, a pharmaceutical additive such as a disintegrant, e.g., starch, agar, alginic acid or its sodium salt, an absorbent, a colorant, a flavorant, a sweetener, etc. The tablet may be prepared by a common mixing, granulation or coating method. In addition, the formulation for parenteral administration may be a formulation for transdermal administration, e.g., an injection, a medicinal drop, an ointment, a lotion, a gel, a cream, a spray, a suspension, an emulsion, a suppository, a patch, etc., although not being limited thereto.

The pharmaceutical composition according to an embodiment of the present disclosure may be administered parenterally, rectally, topically, transdermally, subcutaneously, etc.

Determination of the administration dosage of the active ingredient is within the level of those skilled in the art. A daily administration dosage of the pharmaceutical composition, although it varies depending on various factors such as the severity of a disease to be treated, onset of the disease, age, health condition, complications, etc., may be usually 0.01 to 100 mg/kg/day, e.g., 0.01 mg/kg/day or more, 0.1 mg/kg/day or more, 0.5 mg/kg/day or more, 1 mg/kg/day or more, 1.3 mg/kg/day or more, 1.5 mg/kg/day or more, 1.7 mg/kg/day or more, 2 mg/kg/day or more or 2.2 mg/kg/day or more, and 100 mg/kg/day or less, 50 mg/kg/day or less, 10 mg/kg/day or less, 7 mg/kg/day or less, 5 mg/kg/day or less, 3 mg/kg/day or less or 2.5 mg/kg/day or less, for an adult. Preferably, a daily administration dosage of 1 to 5 mg/kg may be administered once to three times a day. However, the administration dosage does not limit the scope of the present disclosure in any way.

In an embodiment, the composition may be a food composition.

The food composition according to an embodiment of the present disclosure may be a formulation in liquid or solid state, and may be a formulation in the form of a tablet, a capsule, a soft capsule, a pill, a granule, a drink, a diet bar, a chocolate, a caramel or confectionery, although not being specially limited thereto. The food composition of the present disclosure may adequately contain an excipient, a saccharide, a flavorant, a colorant, an oil or fat, a protein, etc. in addition to the active ingredient, if necessary.

Hereinafter, the present disclosure will be described in more detail through the following examples. However, the following examples are provided only for the purpose of illustration to help understanding of the present disclosure, and the scope and range of the present disclosure are not limited thereto.

### EXAMPLES

Camellia japonica seed cake was obtained after producing oil from Camellia japonica seed harvested in Jeju Dongbaek Town through compression. An extract was prepared from the Camellia japonica seed cake using 70% ethanol and it was identified that the Camellia japonica seed cake extract contain terpene saccharides, flavonoid saccharides, sugars and proteins. In particular, it was confirmed that the flavonoid saccharides of the Camellia japonica seed cake consist of kaempferol tetrasaccharides (Chemical Formulas 1 to 4) and trisaccharides (Chemical Formulas 5 to 8).

Purification of Kaempferol Saccharide Fractions of Camellia japonica Seed Cake

After adding 8 kg of 70% ethanol to 1 kg of Camellia japonica seed cake obtained after extracting oil by compression at low temperature, extraction was conducted at 60° C. for 8 hours using a heating extractor. After filtering with a filter press, the obtained extract was concentrated under reduced pressure at 50 to 60° C. with a rotary vacuum evaporator until the solid content reached about 50%, so that 200 g of concentrate was obtained. The concentrate was diluted with purified water such that the solid content became 10%.

After loading the solution in a glass tube column packed with 2 L of an ionexchange resin (HP-20), impurities and sugars were removed by washing with 10 L of purified water. Then, after flowing 10 L of 30% ethanol, the eluate was concentrated under reduced pressure and freeze-dried to obtain about 20 g of Camellia japonica kaempferol saccharide powder

That is, the finally obtained Camellia japonica kaempferol saccharide powder contains both Chemical Formulas 1 to 4 (tetrasaccharides) and 5 to 8 (trisaccharides).

### EXPERIMENTAL EXAMPLES

### Experimental Example 1—DNA Damage Repair Efficacy by Ultraviolet Rays (Evaluation of γ-H2AX Expression)

HaCaT cells were cultured in DMEM medium containing 10% FBS and 1% PS. For immunofluorescence staining, 50,000 cells/well were seeded into 8-well chamber slides. The next day, each cell was pretreated with kaempferol saccharides at the following concentrations for 1 hour. The concentrations of 2.5, 5, and 10 ppm were treated. After 1 hour of pretreatment, the cells were washed with PBS and then irradiated with UVB at 15 mJ/cm². The control group was not irradiated with UVB.

Again, for the group that was not treated with UVB (cont), the group that was irradiated only with UVB, and the group that was irradiates with UVB and was added with kaempferol saccharides, the post treatment was conducted in the same pretreatment conditions. After a total of 24 hours, the cells were harvested for immunofluorescence staining.

For immunofluorescence staining, each well was washed with PBS containing 1 mM CaCl2 and 1 mM MgCl2. The cells were fixed by reacting with 3.5% paraformaldehyde at room temperature for 10 minutes. The fixed cells were washed three times for 10 minutes with PBS again. 0.1% TritonX-100 was treated for 5 minutes. The cells were washed three times for 10 minutes with PBS again. p-H2AX antibody (cell signaling) was diluted 1:100 in PBS (PBST) containing 0.05% Tween 20 and reacted with the primary antibody at 4° C. for O/N. After washing with PBST three times for 10 minutes again, the cells were reacted with secondary antibody conjugated with Rhodamine at 1:200 for 1 hour at room temperature. After that, the cells were washed with PBST three times for 10 minutes. The nuclei were stained with Propidium ipdide (PI) for about 3 minutes. The cells were washed with PBST three times again, added with mounting solution, and covered with coverglass. The stained slides were photographed using a confocal laser scanning microscope (Confocal microscopy, Ziess). Each image was photographed at least 6 times.

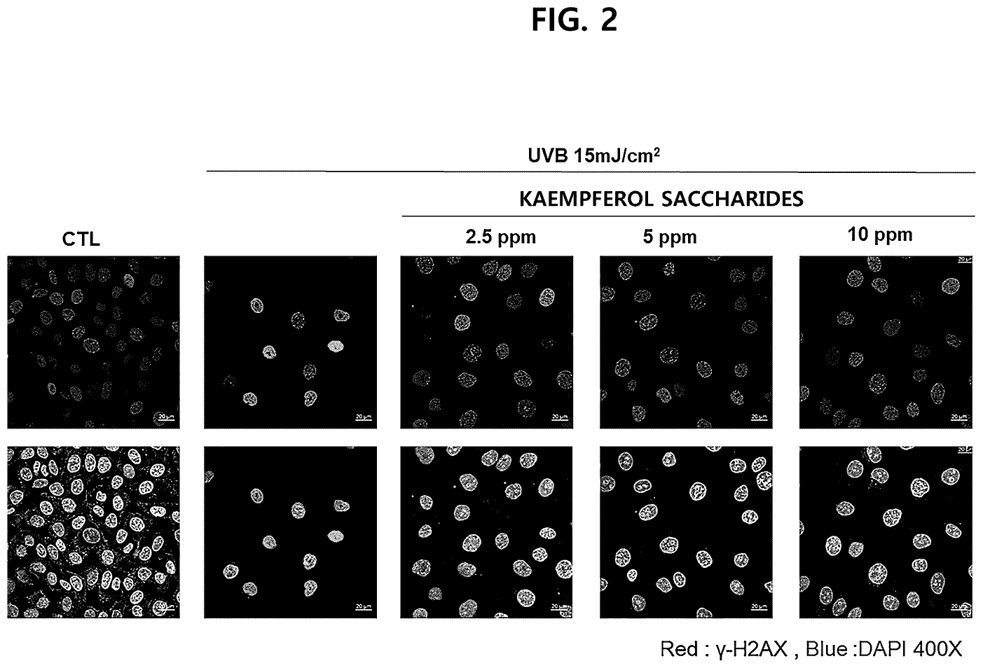

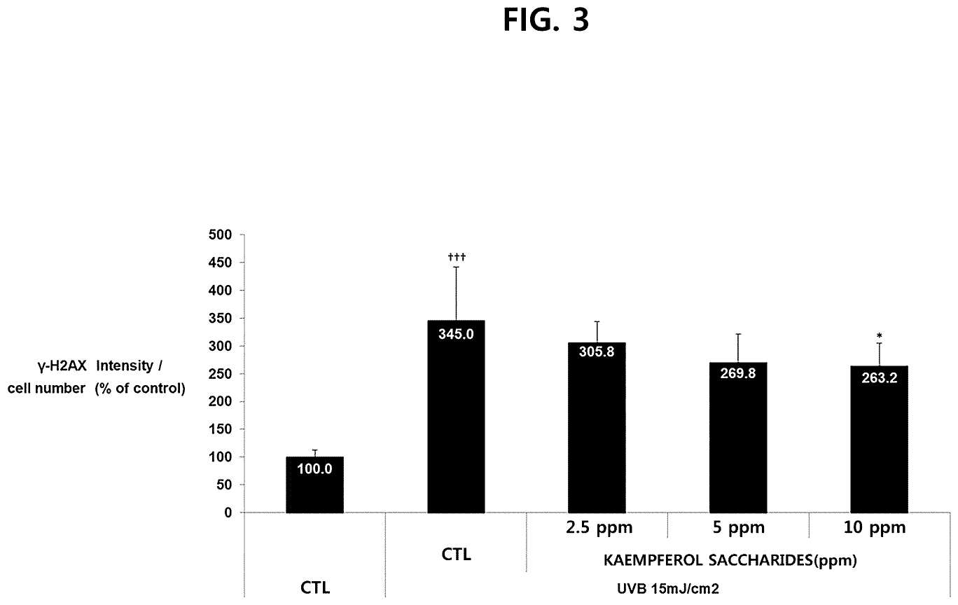

The intensity of each photographed image was measured for each fluorescence using ZEN, which is a confocal-only program. The results are shown in FIGS. 2 and 3, respectively. Specifically, since all fluorescence values are fluorescence counts per cell, the total red signal of one image was measured, and then the number of cells stained with DAPI was measured and calculated, as shown in FIG. 3. (n=6 or more).

As a result, the upper part in FIG. 2 shows only the red signal, which qualitatively confirms that the DNA damage marker γH2AX (red signal) is strongly expressed by UVB and then suppressed in a concentration-dependent manner by kaempferol saccharides. In addition, as shown in the lower part in FIG. 2, it was qualitatively confirmed that the DNA damage marker γH2AX (red signal) was strongly expressed (red-pink color) in the nucleus (same location as the blue signal) by UVB and was suppressed in a concentration-dependent manner by kaempferol saccharides, getting closer to blue color as it went to the right. It was also confirmed that the value decreased through the intensity measurement of γH2AX (red signal) (FIG. 3).

### Experimental Example 2—Cell Protection Against Ultraviolet Rays (Cell Viability)

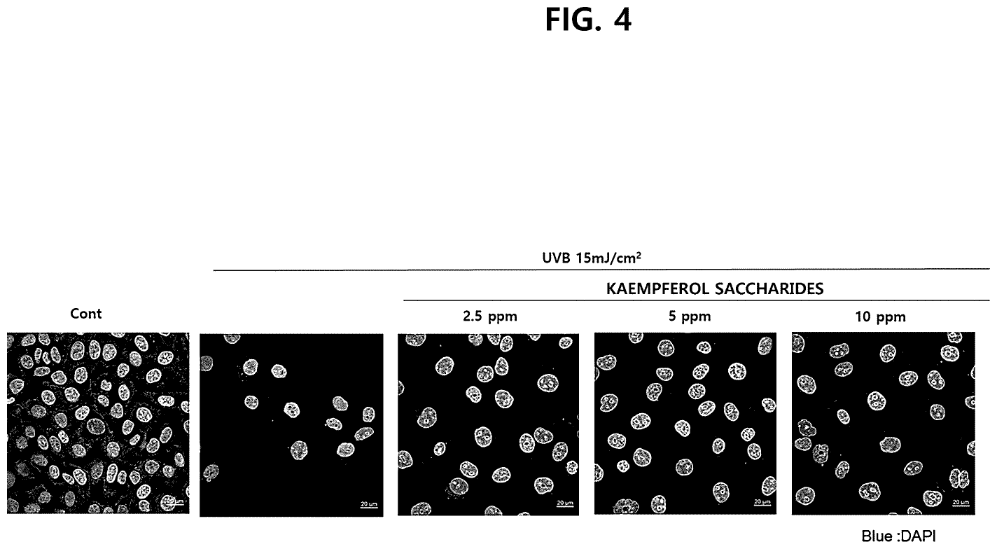

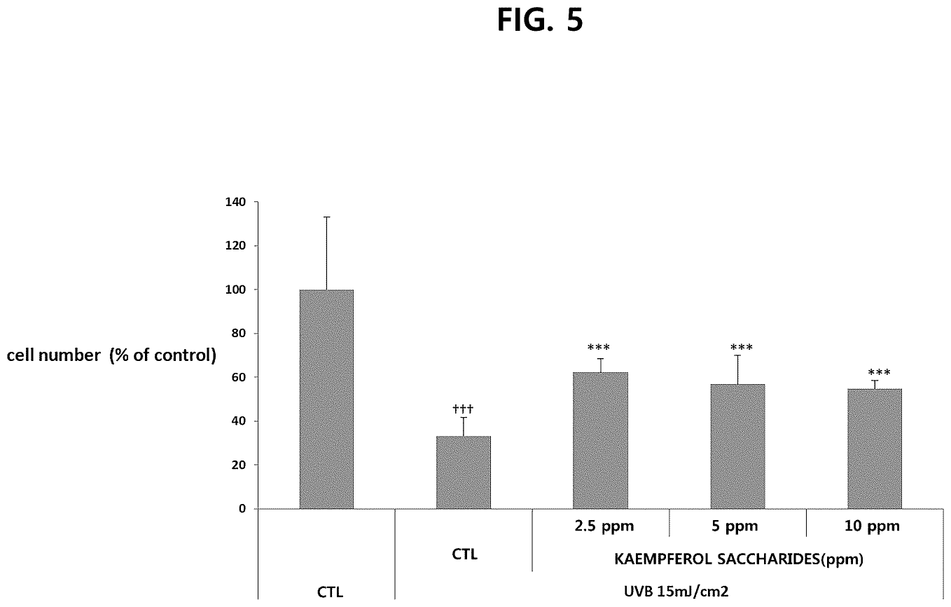

Cell death and growth decline occur due to ultraviolet rays, and the cell protection effect of kaempferol saccharides was observed to overcome this. Specifically, in order to confirm cell survival due to ultraviolet rays, the cell number was directly measured. Only the DAPI image stained in the above Experimental Example 1 was analyzed separately to measure the cell number. The results are shown in FIGS. 4 and 5.

As shown in FIG. 4, the nuclei of living cells were stained with DAPI (blue signal), so the cell number can be directly confirmed with the naked eye. It was confirmed that the cell number decreased due to UVB, and it was confirmed that kaempferol saccharides had the effect of protecting the cell number due to ultraviolet rays. As a result of quantitatively analyzing the cell number (FIG. 5), it was confirmed that the cell number significantly increased. Therefore, the effect of directly increasing cell survival against UVB was confirmed.

What is claimed is:

1. A method for repairing cellular DNA damage, comprising:
administering or applying to a subject in need thereof an effective amount of a kaempferol saccharide compound represented by any one of Chemical Formulas 1 to 8, an isomer thereof, a pharmaceutically acceptable salt thereof, a hydrate thereof, or a solvate thereof:

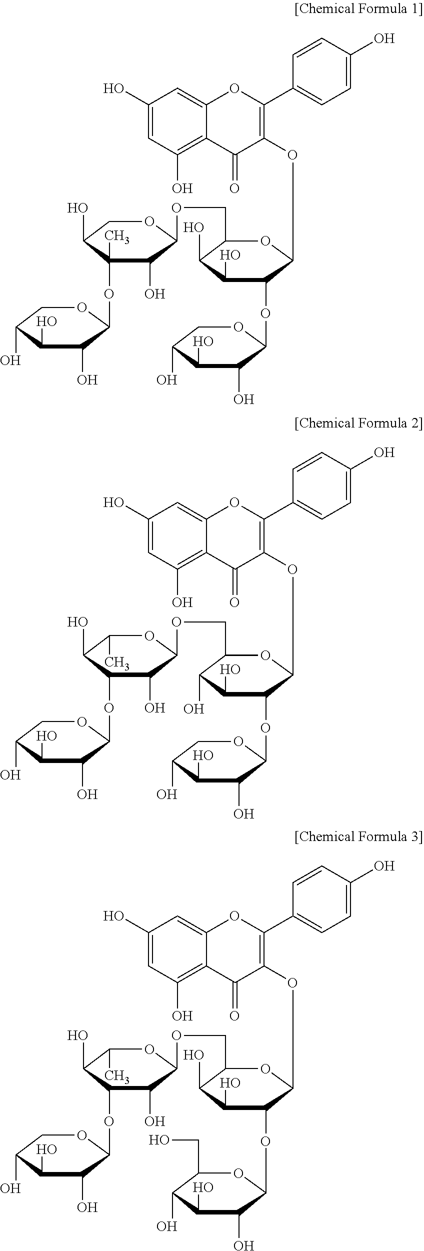

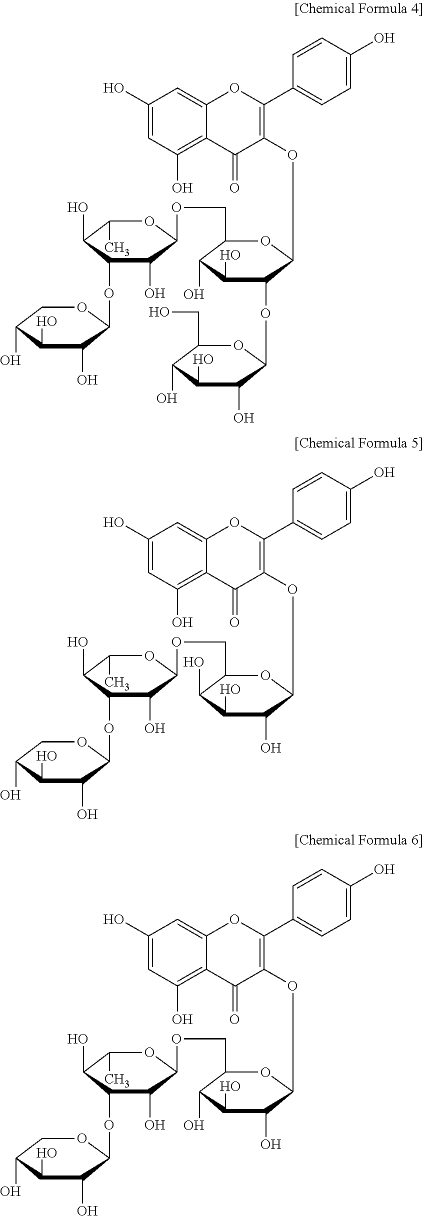

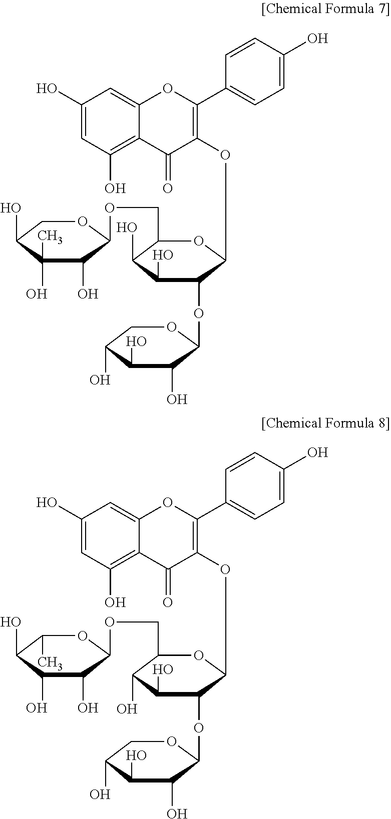

2. The method of claim 1, wherein the kaempferol saccharide compound is extracted from Camellia japonica seed cake.

3. The method of claim 1, wherein a concentration of the kaempferol saccharide compound represented by any one of the Chemical Formulas 1 to 8, an isomer thereof, a pharmaceutically acceptable salt thereof, a hydrate thereof, or a solvate thereof is 0.01 to 100 μM based on the total weight of the composition.

4. The method of claim 1, wherein the cell is a skin cell.

5. The method of claim 1, wherein the composition is for inhibiting or preventing skin damage.

6. The method of claim 1, wherein the composition is for improving skin's natural regenerative power.

7. The method of claim 1, wherein the composition inhibits expression of a DNA damage gene.

8. The method of claim 7, wherein the DNA damage gene is γH2AX.

9. The method of claim 1, wherein the damage is caused by ultraviolet rays.

10. The method of claim 9, wherein the ultraviolet rays are at least one of UVA and UVB.

11. The method of claim 1, wherein the composition is a cosmetic composition.

12. The method of claim 1, wherein the composition is a pharmaceutical composition.

13. The method of claim 1, wherein a dosage or application amount of the kaempferol saccharide compound represented by any one of the Chemical Formulas 1 to 8, the isomer thereof, the pharmaceutically acceptable salt thereof, the hydrate thereof, or the solvate thereof is 0.01 to 100 mg/kg/day.

14. The method of claim 1, wherein the composition is a food composition.
